(19) United States
(12) Patent Application Publication
(10) Pub. No.: US 2007/0106288 A1
(43) Pub. Date: May 10, 2007

(54) Electrosurgical apparatus with fluid flow regulator

(75) Inventors: Jean Woloszko, Mountain View, CA (US); Robert H. Dahla, Sunnyvale, CA (US)

Correspondence Address: ARTHROCARE CORPORATION, 680 VAQUEROS AVENUE, SUNNYVALE, CA, 94085-3523, US

(73) Assignee: ArthroCare Corporation, Austin, TX (US)

(21) Appl. No.: 11/270,344

(22) Filed: Nov. 9, 2005

(51) Int. Cl. A61B 18/14

(52) U.S. Cl. 606/41; 606/45

(57) ABSTRACT

An electrosurgical instrument comprising a shaft that includes a distal end section and a distal tip. Near the distal tip is an active electrode and a fluid collection chamber. The fluid collection chamber comprises an ingress port for suctioning a fluid flow over the active electrode and into the fluid collection chamber; a regulator adapted to adjust the fluid flow through the ingress port; and an aspiration port for exhausting the fluid from the fluid collection chamber, and a method thereof.

### FIELD OF INVENTION

This invention relates to an electrosurgical apparatus and method, in particular an electrosurgical apparatus wherein a fluid regulator on a distal end of a shaft regulates the flow of fluid over an active electrode and into an ingress port on the shaft. In one embodiment the fluid flow into the ingress port is regulated such that the temperature of the electrode is controlled, the plasma generated at the electrode is stabilized, and bubbles formed around the electrode and the target site during the procedure are removed, for better visualization of the electrode and the target site.

### DESCRIPTION OF PRIOR ART

An electrosurgical system as shown for example in FIG. 1 typically comprises an electrosurgical apparatus (10) used in procedures to treat tissue at a target site. The system includes a voltage regulator (12) that provides a high-frequency voltage potential difference cross an active and return electrodes (14) at the tip of a shaft (11), to treat the target site. In treating the target site the electrodes are energized and manipulated to ablate, heat, cut, remove, puncture, probe, brush and otherwise modify tissue at the target site. The target site may include various parts of the body such as the shoulder, skin, knee, nose, spine, neck, hip, heart and the throat.

In treating the target site, the current across the electrodes is applied in several ways, e.g., the current is passed directly into the target site by direct contact with the electrodes such that the current passes into and heats the target site; or the current is passed indirectly into the target site through an electrically conductive fluid located between the electrode and the target site also to heat the target site; or current is passed into an electrically conductive fluid disposed between the electrodes to generate plasma which is used to ablate tissue at the target site. In the procedure wherein plasma is generated, the current does not pass in to the tissue. In various procedures, the conductive fluid is an electrolyte such as isotonic saline and other fluids having conductivity similar to isotonic saline and body fluids. Examples of an electrosurgical apparatus, system and methods of using plasma to treat a target site are described in commonly assigned U.S. Pat. No. 6,149,620 and U.S. patent application Ser. No. 09/457,201, herein incorporated by reference for all purposes.

In using the apparatus (10) to generate plasma to treat tissue in a “wet field” procedure, the tip (14) of the shaft (11) comprising the active electrode is placed in a conductive fluid on the target site. For the present purposes, a wet field procedure is a procedure wherein the target site is flooded with a conductive fluid. With reference to FIG. 2, which illustrates an expanded view of a tip of an embodiment of the shaft (11), the tip comprises a distal end (13) that includes an irrigation fluid lumen (17) integrated into the shaft. In various embodiments the irrigation lumen is connected to a conductive fluid supply (18) as illustrated in FIG. 1, for supplying the conductive fluid. Additionally, an aspiration lumen (20) is provided for removing fluids from the target site (19). In a wet field procedure, the conductive fluid forms an electrically conductive layer or a conductive fluid bridge between the active electrode (15) and the return electrode (26). On application of a high frequency voltage potential across the electrodes, ions within the conductive fluid are energized to from plasma between the electrodes (15, 26). As used herein, an active electrode is an electrode that is adapted to generate a higher charge density, and hence generate more plasma, relative to a return electrode when a high-frequency voltage potential is applied across the electrodes. Typically, a higher charge density is obtained by making the active electrode surface area smaller relative to the surface area of the return electrode.

With reference to FIG. 2, in one embodiment the distal end (13) of the shaft comprising the irrigation lumen (17) terminates at a discharge port (24a) located near the active electrode (15). Additionally, in other embodiments a suction lumen (20) that originates at an aspiration port (24b) located near the return electrode (26) is provided to remove fluids and ablated tissue from the target site. In the embodiment illustrated in FIG. 2, the active electrode (15) is spaced apart from the return electrode (26) by an insulating spacer (28). In this embodiment, the spacer (28) is formed with a spacer lumen (28a) such that when the spacer is in position on the shaft, its lumen is aligned transversely across the distal end of the shaft (13) such that the target site (19) is visible from above the shaft through the lumen. An example of such an apparatus and a procedure for treating a target site with this apparatus are described in commonly assigned U.S. patent application Ser. No. 10/661,118, supra, herein incorporated by reference for all purposes.

With reference to FIG. 2, a problem that occurs with the apparatus during use in a wet field is that visualization of the target site (19) and the active electrode (15) is impaired due to gas bubbles (30) forming at the electrode (15) and at the target site (19). The bubbles are formed from gases derived from the conductive fluid, and/or from disintegrated body tissue at the target site. As the bubbles are hot and buoyant, they rise and form a plume over the target site and the distal tip of the shaft (13), causing the visual impairment. Thus it is desirable to remove the bubbles or at least control their formation such that visualization of the site and the electrode is not compromised.

In the prior art, one possible approach to removing the bubbles from the target site is to increase the fluid flow to the site, while simultaneously suctioning off the fluid from the site at a rate such that the bubbles are captured in the fluid flow. While this approach will remove bubbles, an undesirable consequence of the increase fluid flow across the electrode is that the temperature of the electrode is lowered, which has the undesirable effect of decreasing the stability of the plasma generated. Thus, with this approach, in order to maintain the stability of the plasma, the current through the electrodes is increased to maintain the temperature of the electrode at the desired plasma-generating temperature level.

However, on increasing the in current to the electrode, besides increasing the temperature of the electrodes, the temperature of the conductive fluid around the electrode also increases, which has the undesirable consequence of increasing the risk of burns to the patient and heat damage to the tissue.

Accordingly, in view of the above disadvantages of in the prior art, there is a need for a better way to stabilize the plasma at the electrodes, and also to control bubbles at the target site, without increasing the risk of heat damage to the tissue, or burns to the patient. It is thus an objective of the present invention to address these needs.

### SUMMARY OF THE INVENTION

In one embodiment, the present apparatus is an electrosurgical instrument comprising: a shaft comprising a distal end section including a distal tip; and an active electrode disposed near the distal tip, wherein the distal end section comprises a fluid collection chamber. In one embodiment the fluid collection chamber comprises an ingress port for suctioning a fluid flow over the active electrode and into the fluid collection chamber; a regulator adapted to adjust the fluid flow through the ingress port; and an aspiration port for exhausting the fluid from the fluid collection chamber.

In another embodiment, the apparatus is an electrosurgical instrument for treating a target site comprising: a shaft comprising a distal end section, a distal tip, and a fluid aspiration lumen extending to the distal tip. On the distal end is a fluid collection chamber in fluid communication with the aspiration lumen, the fluid collection chamber comprising: a fluid ingress port such that fluid in the vicinity of the target site may be drawn therein at a first flowrate, and transported into the aspiration lumen; and a regulator, the regulator adapted to adjust the first flowrate such that the first flowrate is independent of a third flowrate through the aspiration lumen; and an active electrode arranged at the distal end section such that fluid entering the ingress port is drawn across the active electrode.

In various embodiments of the apparatus, the fluid ingress port comprises a first cross section area, and the regulator comprises a second cross section area such that the ratio of the second cross section area to the first cross section area is equal to or greater than about ⅗; in another embodiment the ratio of the second cross section area to the first cross section area is equal to or greater than about 1; while in a further embodiment the ratio of the second cross section area to the first cross section area is equal to or greater than about 3/2. In one embodiment, the second section area is about 0.0030 square inch to about 0.0050 square inch. In various embodiments the regulator comprises one or more openings formed into the fluid collection chamber; in one embodiment the regulator comprises one more valves.

In another embodiment, the present method comprises performing an electrosurgical procedure on a target site, including the steps of: applying a high-frequency voltage potential difference between an active electrode and a return electrode of an electrosurgical apparatus in the presence of an electrically conductive fluid, in close proximity to the target site; removing a first fluid stream from the target site through an ingress port on the electrosurgical apparatus, at a first flow rate, wherein the first fluid stream comprises fluids in contact with the active electrode; suctioning a second fluid stream from said target site through a regulator on the apparatus; wherein the first fluid stream flow is regulated by the second stream flow and bubbles at the target site are removed for improved visualization of the target site during the procedure.

Advantageously, with the present apparatus and method, since the flow of fluid through the ingress port and across the active electrode is regulated by the fluid flow through the regulator, the bubbles generated at the electrode and target site are removed, without increasing the fluid flow across the active electrode. Consequently, with the present apparatus and method, the plasma at the active electrode is stabilized without increasing the current through the electrodes. Also, because the current through the electrodes is not increased, heating of the electrode is not increased, and therefore the risk of causing thermal injury to the patient is not increased.

Embodiments of the present apparatus and methods are illustrated schematically in the following Figures, and described in greater detail in the following sections of the specification.

### BRIEF DESCRIPTION OF THE DRAWINGS

FIG. 1 is an illustration of an electrosurgical apparatus and system for treating target sites in the body.

FIG. 2 is an illustration of a prior art apparatus wherein bubbles generated at the distal end the apparatus impair visualization of the electrode and the target site.

FIG. 3A is an illustration of an embodiment of the present apparatus wherein bubbles at the distal end are collected in a fluid collection chamber and removed from the target site, to improve visualization.

FIG. 3B is an illustration of embodiment of the present apparatus wherein a plurality of ingress ports are provide at the distal end of a shaft for regulating the flow of fluid into a fluid collection chamber.

FIG. 3C is an illustration of an embodiment of the present apparatus wherein an active electrode is provided across a fluid ingress port for generating plasma to treat a target site.

### DETAILED DESCRIPTION

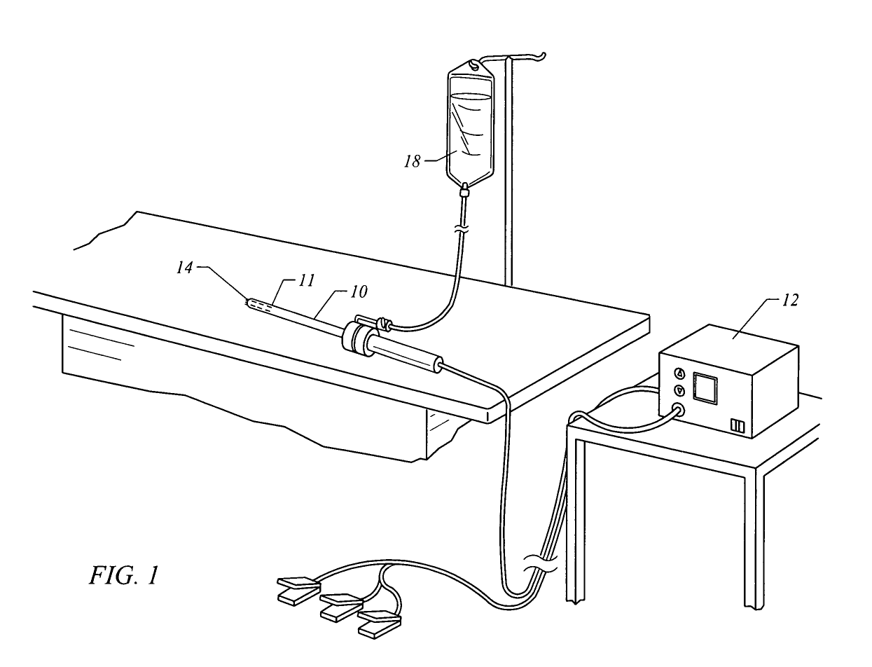

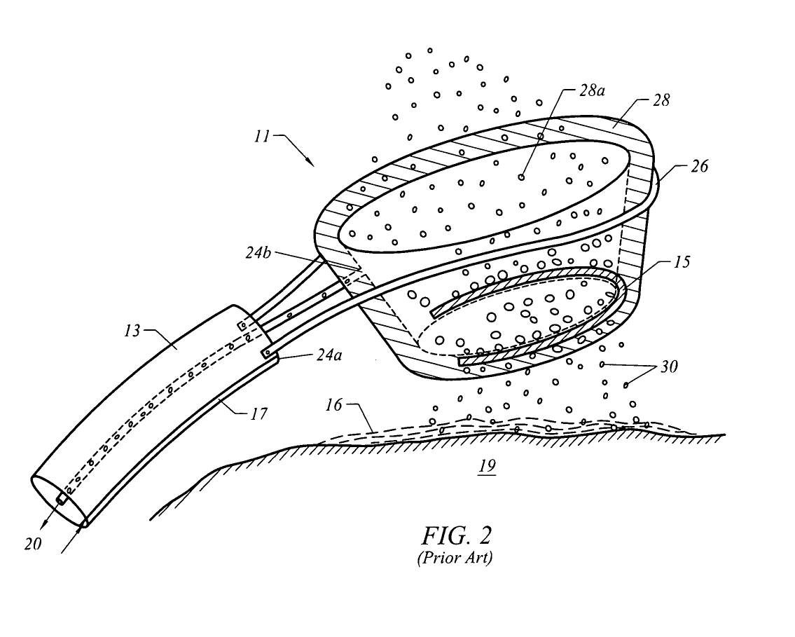

The following description of preferred embodiments of the apparatus and method is provided in conjunction with the illustrations of FIGS. 1-3C. However, it will be appreciated by one ordinarily skilled in the art that the present apparatus and method can be described and practiced with modifications and variations that are well within the scope of the appended claims.

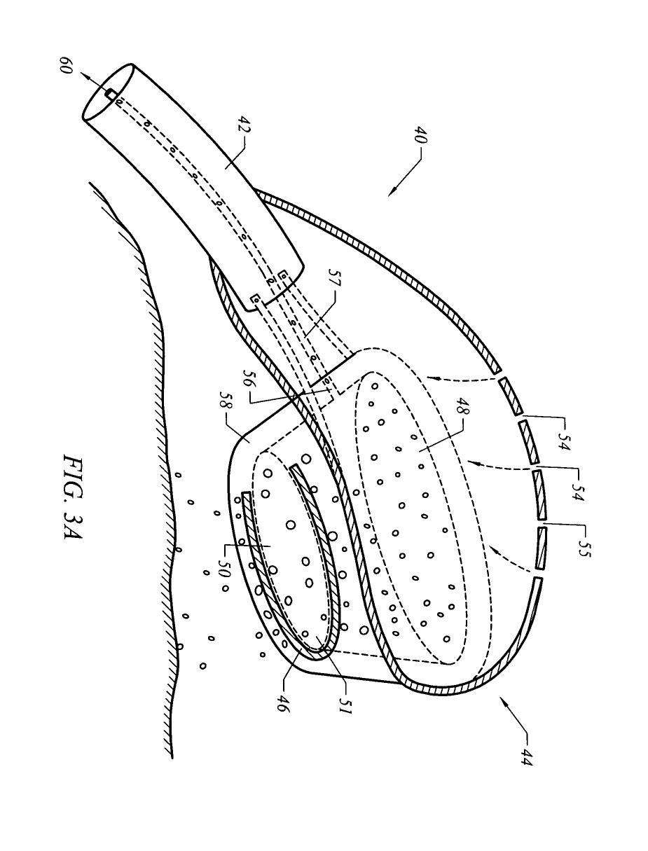

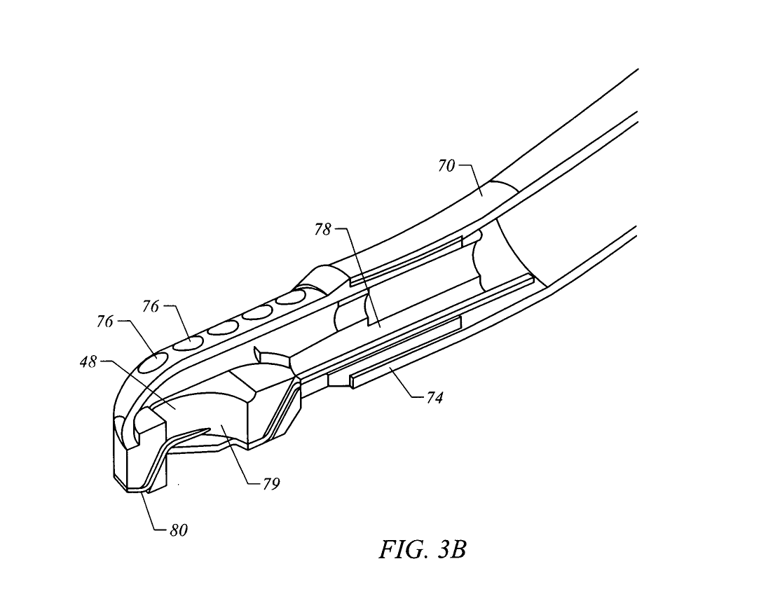

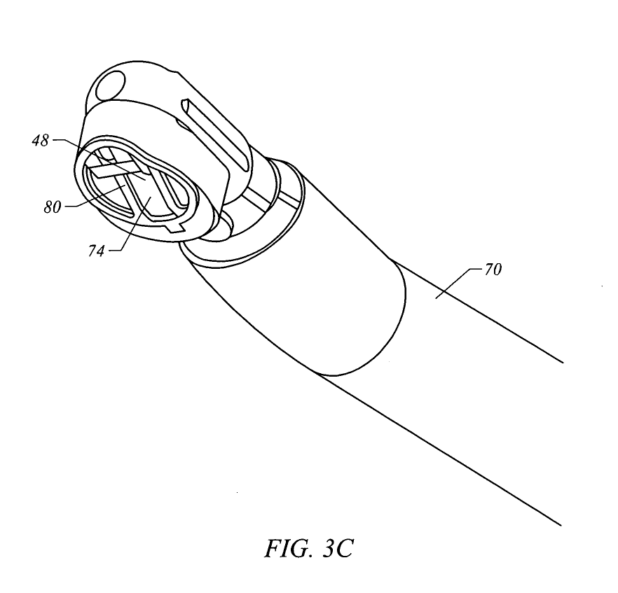

With reference to FIG. 3A, the apparatus (40) in one embodiment comprises a shaft (42) having a distal end that includes a distal tip(44); an active electrode (46) disposed at the distal end; and a fluid collection chamber (48) located at the distal end. In various embodiments, the shaft and the active electrode are conventional and are described in greater detail for example in commonly assigned U.S. Pat. No. 6,149,620 and U.S. patent application Ser. No. 09/457,201, herein incorporated by reference for all purposes.

With reference to FIG. 3A, the fluid collection chamber (48) in one embodiment is shaped in the form of cap that is inserted axially on the distal end of the shaft, and comprises an ingress port (50), a fluid regulator comprised of a plurality of holes (54) into the chamber, and an aspiration port (56) that together cooperate to control the flow of fluid over the active electrode (46) into the ingress port. In another embodiment the cap is in the form of a sleeve comprised of the ingress port (50), the fluid regulator (54), and the aspiration port (56) that together cooperate to control the flow of fluid across the active electrode (46) through the ingress. In still another embodiment the fluid regulator comprises one or more valves through which fluid flow into the fluid collection chamber is regulated.

In one embodiment the ingress port (50) is provided with a first cross-section area (51) for suctioning fluids from the target site (52) into the fluid collection chamber. Deployed across the ingress port, or at least partly circumscribing the ingress port, is an active electrode (46). In this embodiment, the regulator (54) is designed to allow entry of fluid into the fluid chamber, and comprises one or more openings (54) spaced away from the ingress port.

In various embodiments the fluid ingress port (50) comprises a first cross section area, and the regulator comprises a second cross section area such that the ratio of the second cross section area to the first cross section area is equal to or greater than about ⅗; in another embodiment this ratio is equal to or greater than about 1, while in a further embodiment this ration is equal to or greater than about 3/2. In one embodiment the cross section area of the second opening is in the range of about 0.0030 square inch to about 0.0050 square inch. Thus, in this embodiment, since the volume of the fluid chambers fixed, therefore fluid flow through the regulator can be adjusted to regulate the flow of fluid through the ingress port and across the active electrode. Under normal operating conditions, the above-noted ratio has been found to provide sufficient fluid flow across the active electrode such that the plasma is stabilized, the temperature of the fluid is controlled, and bubbles are removed without the need to increase the current through the electrodes.

In various embodiments an aspiration port (56) having a third cross-section area (57) for aspirating and exhausting fluids from the fluid collection chamber is provided. In one embodiment, the aspiration port is connected to a vacuum system (not shown) for evacuating fluid from the collection chamber.

The fluid cap or sleeve in various embodiments is comprised of conventional material as, for example, the conductive material of the shaft; in alternative embodiments the material is non-conductive as, for example, a polymer or a ceramic. In one embodiment the fluid cap or sleeve is adapted to function as a return electrode; in this embodiment, the fluid cap as illustrated in FIG. 3A, is insulated from the active electrode by spacer (58), and is connected to a high frequency power supply comprising the active electrode and a conductive fluid present on the target site. In one embodiment not illustrated, the fluid collection chambers comprise an axial lumen formed in the distal end of the shaft; in another embodiment not illustrated the fluid collection chamber comprises a fluid chamber positioned on the distal end of the shaft.

As is illustrated in FIG. 3A, in one embodiment a spacer is attached at the distal end of the shaft (42) and defines a spacer lumen therein that is generally transverse to the axial orientation of the shaft, and is located between the active electrode (46) and the fluid cap (48). In one embodiment the spacer also defines an aspiration port (56) connected to a vacuum system through a vacuum lumen (60) in the shaft (42). In various embodiments the spacer comprises a non-conductive material such as a plastic or a ceramic.

In a preferred embodiment as illustrated in FIGS. 3A, 3B and 3C, the regulator comprises a plurality of openings (54) into the fluid collection chamber (48). In this embodiment, the regulator cross-section area comprises the sum of the cross-section areas of the plurality of openings. In an embodiment not illustrated, the openings are provided with a plurality of adjustable valves that permit inflow of fluid into the fluid collection chamber, but prevent the outflow of fluids including bubbles through the openings. An example of such a valve is a conventional flapper-type valve commonly known in the art.

Without desiring to be bound by any theory pertaining to the results achieved by the present apparatus and method, it is believed that because the holes of the regulator into the fluid collection chamber are either as small as and or smaller than the bubbles, the bubbles are prevented from escaping through the regulator. In one embodiment as illustrated in Table 1, the holes are sized to provide an opening of about 0.0030 square inch to about 0.0050 square inch into the collection chamber. As is illustrated schematically in FIGS. 3A and 3B, in a preferred embodiment the holes of the regulator are located away from the ingress port and the active electrode (46, 80) such that the regulator can be use to throttle the flow of fluid through the ingress port. Further with the present apparatus, since the opening of the regulator can be adjusted, an adjustment can b make to maintain a steady state pressure drop across the inlet port and the collection chamber

In experiments conducted to with the present apparatus to determine the stability of the plasma at the electrodes for various first and second cross-section areas of the present apparatus and fluid flow, it was observed that sufficient stable plasma forms when the ratios of the second cross-section area to the first cross-section area equal to or greater than about 1, and in particular to a ratio equal to or less than about 3/2. A summary of the experiments results is provided in Table 1.

TABLE 1
First and Second cross-section areas of
ports on the Fluid Collection Chamber

| Ingress Port | | | |
|---|---|---|---|
| Area (first | No. of holes | Regulator (second | |
| cross-section | in collection | cross-section area) | Plasma formed on |
| area) (in²) | chamber | (in²) | electrode? |
| 0.005 | 6 | 0.0030 | Yes |
| 0.005 | 7 | 0.0035 | Yes |
| 0.005 | 8 | 0.0040 | Yes |
| 0.005 | 9 | 0.0045 | Yes |
| 0.005 | 10 | 0.0050 | Yes |

In various embodiments, the fluids aspirated from the target site through the ingress port comprise gas bubbles, water vapor, conductive fluids, disintegrating body tissue, bone fragments and body fluids. In one procedure, as illustrated in FIG. 3A, fluid is supplied to the site through a flushing lumen (24a) located at the distal end of the shaft. Typically, the flushing fluid is an electrically conductive fluid such as isotonic saline and its equivalent. In another procedure the fluid is derived from body fluids and disintegrating tissue at the target site.

In another embodiment the present apparatus as illustrated for example in FIG. 3B comprises a shaft (70) having a distal end; an aspiration lumen (72) disposed at the distal end of the shaft and terminating in an ingress port (74) for suctioning fluids into the aspiration lumen, a regulator ports (76) for regulating flow of the fluids into the aspiration lumen, and an aspiration port (78) for exhausting fluids from the aspiration lumen. As with the alternative embodiment described above, the regulator port is adapted for regulating flow of fluids into the ingress port, and comprises perforations having a cross-section area wherein a ratio of the perforation cross-section area to the ingress port cross-section area is equal to or greater than about ⅗. In this embodiment, the apparatus includes an active electrode (80) disposed near the ingress port, and a return electrode on the shaft that is connected to a high frequency power supply. In this embodiment the aspiration lumen (72) is connected to a vacuum system, not shown in the Figures.

In one embodiment the present method is a procedure of performing an electrosurgical procedure on tissue at a target site and removing bubbles that impair visualization of the target site, comprising applying a voltage potential difference between an active electrode of an electrosurgical apparatus in close proximity to the target site and a return electrode in the presence of an electrically conductive fluid on the target site; aspirating a first stream of material from the target site through a fluid ingress port of the apparatus at a first flow rate; suctioning a second stream of electrically conductive from the target site through a regulator of the apparatus; whereby the first flow rate is regulated by the suctioning step, thereby treating the target site and removing bubbles that impair visualization of the target site.

In one embodiment first flow rate into the fluid chamber and across the active electrode through the ingress port is regulated such that it is substantially constant. In accordance with the present apparatus the constant flow rate is achieved by dimensioning the ingress port to have a first cross-section area, and the ingress port to have a second cross-section area such that the ratio of said second cross-section area to said first cross-section area is equal to or greater than about ⅗. In other embodiments the ratio of the second cross-section area to first cross-section area is equal to or greater than about 1, and in a preferred embodiment the ratio of the second cross-section area to said first cross-section area is equal to or less than about 3/2. As is illustrated in Table 1 and described above, the second cross-sectional area is sized for an opening of about 0.0030 square inch to about 0.0050 square inch in the apparatus.

In various embodiments, the method further comprises aspirating the bubbles from the fluid collection chamber to maintain visualization of the target site. As will be appreciated by one ordinarily skilled in the art, the present method may be used to treat target tissue includes ablating, puncturing, and cutting the target tissue. Depending on the tissue being treated, in one procedure a voltage of about 50 volts to 1000 volts can be applied; in other procedures, a voltage in the range of 200 volts to 350 volts can be applied. In various embodiment treatment include directing a conductive fluid to the target tissue so as to ablate, puncture, and volumetrically remove tissue.

While the invention is described with reference to the Figures and method herein, it will be appreciated by one ordinarily skilled in the art that the invention can also be practiced with modifications that are within the scope of the claims. Thus the scope of the invention should not be limited to the embodiments as described herein, but is limited only by the scope of the appended claims.

What is claimed is:

1. An electrosurgical apparatus for treating a target site, comprising:
a shaft comprising a distal end section including a distal tip; and
an active electrode disposed near the distal tip,
wherein
the distal end section comprises a fluid collection chamber comprising:
an ingress port for suctioning a fluid flow over the active electrode and into the fluid collection chamber;
a regulator adapted to adjust the fluid flow through the ingress port; and
an aspiration port for exhausting the fluid from the fluid collection chamber.

2. The electrosurgical apparatus of claim 1, wherein the fluid collection chamber comprises a lumen extending through the distal end section of shaft.

3. The electrosurgical apparatus of claim 1, wherein the fluid collection chamber comprises a sleeve disposed on the distal end section of the shaft.

4. The electrosurgical apparatus of claim 1, further comprising a return electrode arranged at the distal end section of the shaft.

5. The electrosurgical apparatus of claim 1, wherein the ingress port comprises an opening into the fluid collection chamber near the active electrode.

6. The electrosurgical apparatus of claim 1, wherein the ingress port is partly surrounded by the active electrode.

7. The electrosurgical apparatus of claim 1, wherein the ingress port is partly covered by the active electrode.

8. The electrosurgical apparatus of claim 1, wherein the regulator comprises one or more openings formed into the fluid collection chamber.

9. The electrosurgical apparatus of claim 1, wherein the regulator comprises one more valves.

10. The electrosurgical apparatus of claim 1, wherein the regulator is adjustable for regulating fluid flow into the collection chamber.

11. The electrosurgical apparatus of claim 1, wherein the aspiration port comprises a lumen formed through the distal end section of the shaft.

12. The electrosurgical apparatus of claim 1, wherein said active electrode is connectable to a high frequency voltage regulator.

13. The electrosurgical apparatus of claim 1, comprising an electrical insulator disposed between the active electrode and the fluid collection chamber.

14. The electrosurgical apparatus of claim 13, wherein the active electrode is partly embedded in the electrical insulator.

15. The electrosurgical apparatus of claim 13, wherein the ingress port comprises a lumen formed through the insulator.

16. The apparatus of claim 1, wherein the fluid ingress port comprises a first cross section area, and the regulator comprises a second cross section area such that the ratio of the second cross section area to the first cross section area is equal to or greater than about ⅗.

17. The apparatus of claim 17, the ratio of the second cross section area to the first cross section area is equal to or greater than about 1.

18. The apparatus of claim 17, wherein the ratio of the second cross section area to the first cross section area is equal to or greater than about 3/2.

19. The apparatus of claim 17, wherein the second cross section area is about 0.0030 square inch to about 0.0050 square inch.

20. An electrosurgical apparatus for treating a target site comprising:
a shaft comprising a distal end section, a distal tip, and a fluid aspiration lumen extending to the distal tip;
a fluid collection chamber arranged at the distal end section, said fluid collection chamber in fluid communication with said aspiration lumen, said fluid collection chamber comprising a fluid ingress port such that fluid in the vicinity of the said target site may be drawn therein at a first flowrate, and a regulator through which fluid enters said fluid collection chamber at a second flowrate, said regulator adapted to adjust said first flowrate such that the first flowrate is independent of a third flowrate through the aspiration lumen; and
an active electrode arranged at said distal end section such that fluid entering said ingress port is drawn across said active.

21. The apparatus of claim 21, wherein the fluid ingress port comprises a first cross section area, and the regulator comprises a second cross section area such that the ratio of the second cross section area to the first cross section area is equal to or greater than about ⅗.

22. The apparatus of claim 21 wherein the ratio of the first cross-section is equal to or greater than about 1.

23. The apparatus of claim 21, wherein the ratio of the second cross section area to the first cross section area is equal to or greater than about 3/2.

24. The apparatus of claim 21, wherein the cross section of the second opening is about 0.0030 square inch to about 0.0050 square inch.

25. The apparatus of claim 20, further comprising a return electrode arranged on the distal end of the shaft.

26. The apparatus of claim 20, wherein the regulator comprises a valve.

27. The apparatus of claim 20, wherein the regulator comprises one or more openings into the fluid collection chamber.

28. A method of performing an electrosurgical procedure on a target site, comprising:
applying a high-frequency voltage potential difference between an active electrode and a return electrode of an electrosurgical apparatus in the presence of an electrically conductive fluid, in close proximity to the target site;
removing a first fluid stream from the target site through an ingress port on the electrosurgical apparatus, at a first flow rate, wherein the first fluid stream comprises fluids in contact with the active electrode;
suctioning a second fluid stream from said target site through a regulator on the apparatus; wherein the first fluid stream flow is regulated by the second stream flow and bubbles at the target site are removed for improved visualization of the target site during the procedure.

29. The method of claim 28, wherein the regulator and the ingress port are sized such that fluid flow through the ingress port is stabilized during the procedure.

30. The method of claim 28, wherein the active electrode spans the ingress port.

31. The method of claim 28, wherein the active electrode circumscribes the ingress port.

32. The method of claim 28, including removing bubbles engulfing the target site by removing the first fluid stream.

33. The method of claim 28, including exhausting fluid from the apparatus through an aspiration port on the apparatus.

34. The method of claim 28, including connecting the aspiration port to a vacuum system.

35. The method of claim 28, including regulating the first fluid stream by adjusting the size of the opening of the regulator.

36. The method of claim 28, including forming stabilized plasma at the active electrode upon applying the high-frequency voltage potential to the active electrode.

37. The method of claim 28, including directing the plasma to treat the target site during the procedure.

38. The method of claim 28, including applying the high-frequency voltage potential at the active electrode to remove volumetric amounts of tissue at the target site.

39. The method of claim 28, including providing an electrically conductive fluid to the target site.

40. The method of claim 28, wherein the regulator comprises a valve.

41. The method of claim 28, wherein the regulator comprises a plurality of holes into the apparatus.

42. The method of claim 28, wherein the regulator cross-section area is about 0.0030 square inch to about 0.0050 square inch.

43. The method of claim 28, wherein the fluid comprises bubbles, water vapor, electrically conductive fluids, body tissue, and bone fragments.
